(19) United States
(12) Patent Application Publication
(10) Pub. No.: US 2024/0271048 A1
(43) Pub. Date: Aug. 15, 2024

(54) Processes for Dehydrogenating Alkane and Alkyl Aromatic Hydrocarbons

(71) Applicant: ExxonMobil Chemical Patents Inc., Baytown, TX (US)

(72) Inventor: Xiaoying Bao, Houston, TX (US)

(21) Appl. No.: 18/566,981

(22) PCT Filed: May 25, 2022
(86) PCT No.: PCT/US2022/030862

§ 371 Date: Dec. 4, 2023

Related U.S. Application Data
(60) Provisional application No. 63/202,590, filed on Jun. 17, 2021.

(51) Int. Cl. C10G 11/02; B01J 21/04; B01J 21/08; B01J 23/26; B01J 23/42; B01J 23/58; B01J 35/00

(52) U.S. Cl. CPC C10G 11/02; B01J 21/04; B01J 21/08; B01J 23/26; B01J 23/42; B01J 23/58; B01J 35/19; C10G 2300/1037; C10G 2300/4006; C10G 2300/4012; C10G 2300/4018; C10G 2300/706

(57) ABSTRACT

Processes for converting an alkane to an alkene. In some embodiments, the process can include contacting a hydrocarbon-containing feed with a first catalyst that can include Pt or a second catalyst that can include Cr within a conversion zone to effect dehydrogenation of at least a portion of the hydrocarbon-containing feed to produce an effluent that can include one or more dehydrogenated hydrocarbons and molecular hydrogen. The process can also include contacting the effluent with a solid oxygen carrier disposed within the conversion zone to effect combustion of at least a portion of the molecular hydrogen to produce a conversion product that can include the one or more dehydrogenated hydrocarbons and water. In some embodiments, contacting the feed with the first or second catalyst can occur in a first conversion zone and contacting the effluent with the solid oxygen carrier can occur in a second conversion zone.

### CROSS-REFERENCE TO RELATED APPLICATION

This application claims priority to and the benefit of U.S. Provisional Application No. 63/202,590 having a filing date of Jun. 17, 2021, the disclosure of which is incorporated herein by reference in its entirety.

### FIELD

This disclosure relates to processes for dehydrogenating alkane and/or alkyl aromatic hydrocarbons. More particularly, this disclosure relates to processes for dehydrogenating alkane and/or alkyl aromatic hydrocarbons in the presence of a first catalyst or a second to produce an effluent that includes a dehydrogenated product and molecular hydrogen and combusting the molecular hydrogen in the presence of a solid oxygen carrier to produce a conversion product that includes the dehydrogenated hydrocarbon and water.

### BACKGROUND

Dehydrogenation is an industrially important chemical conversion process that is endothermic and equilibrium-limited. The dehydrogenation of alkanes, e.g., C₂-C₁₂ alkanes, and/or alkyl aromatics, e.g., ethylbenzene, can be done through a variety of different catalyst systems such as the Pt-based, Cr-based, Ga-based, V-based, Zr-based, In-based, W-based, Mo-based, Zn-based, Fe-based systems. In order to enhance equilibrium conversion, reduced operating pressure, hydrocarbon feed dilution, and/or increased operating temperature are often employed that introduce extra operating costs, promote undesirable side reactions, and/or lead to catalyst deactivation.

An alternative to shift chemical equilibrium toward the desired dehydrogenated product is to mix the catalyst used in the dehydrogenation process with a metal oxide (“solid oxygen carrier” or “SOC”) with multiple redox states at the relevant reaction conditions. Molecular hydrogen that is produced during the processes can then be combusted via the lattice oxygen in the solid oxygen carrier. The combustion of molecular hydrogen, however, produces water, which may cause premature deactivation of the dehydrogenation catalyst. Furthermore, frequent regeneration of the catalyst system using an oxygen-containing gas to replenish the lattice oxygen in the solid oxygen carrier requires that the dehydrogenation catalyst be stable against such oxidative regeneration. In addition, it is desirable that the dehydrogenation catalyst be active in the absence of a pre-reduction step, e.g., contact with molecular hydrogen under sufficient conditions, to avoid stripping lattice oxygen from the solid oxygen carrier.

There is a need, therefore, for improved processes for dehydrogenating alkane and/or alkyl aromatic hydrocarbons. This disclosure satisfies this and other needs.

### SUMMARY

Processes for dehydrogenating alkane and/or alkyl aromatic hydrocarbons are provided. In some embodiments, the process can include (I) feeding a hydrocarbon-containing feed that can include one or more of C₂-C₁₆ linear or branched alkanes, one or more of C₄-C₁₆ cyclic alkanes, one or more of C₈-C₁₆ alkyl aromatics, or a mixture thereof into a conversion zone. The process can also include (II) contacting the hydrocarbon-containing feed with a first catalyst that can include Pt disposed on a first support or a second catalyst that can include Cr disposed on a second support within the conversion zone to effect dehydrogenation of at least a portion of the hydrocarbon-containing feed to produce an effluent comprising one or more dehydrogenated hydrocarbons and molecular hydrogen. The first catalyst, if present, can include 0.025 wt % to 6 wt % of Pt based on a total weight of the first support. The first support can include at least one of: (i) at least one compound that can include at least one metal having an atomic number of 21, 39, or 57-71 and at least one compound that can include at least one Group 4, 5, 6, 12, 13, 14, 15, or 16 metal or metalloid, and (ii) at least one compound that can include at least one metal having an atomic number of 21, 39, or 57-71 and at least one Group 4, 5, 6, 12, 13, 14, 15, or 16 metal or metalloid. A molar ratio of the at least one metal having the atomic number of 21, 39, or 57-71 to the at least one Group 4, 5, 6, 12, 13, 14, 15, or 16 metal or metalloid can be at least 0.03:1. A molar ratio of the at least one metal having the atomic number of 21, 39, or 57-71 to the Pt can be at least 30:1. The second catalyst, if present, can include 0.025 wt % to 50 wt % of Cr based on a total weight of the second support. The second support can include SiO₂, ZrO₂, TiO₂, or a mixture thereof. The process can also include (III) contacting the effluent with a solid oxygen carrier disposed within the conversion zone to effect combustion of at least a portion of the molecular hydrogen to produce a conversion product that can include the one or more dehydrogenated hydrocarbons and water.

In other embodiments, the process for dehydrogenating a hydrocarbon can include (I) feeding a hydrocarbon-containing feed that can include one or more of C₂-C₁₆ linear or branched alkanes, one or more of C₄-C₁₆ cyclic alkanes, one or more of C₈-C₁₆ alkyl aromatics, or a mixture thereof into a first conversion zone. The process can also include (II) contacting the hydrocarbon-containing feed with a first catalyst that can include Pt disposed on a first support or a second catalyst that can include Cr disposed on a second support within the first conversion zone to effect dehydrogenation of at least a portion of the hydrocarbon-containing feed to produce an effluent that can include one or more dehydrogenated hydrocarbons and molecular hydrogen. The first catalyst, if present, can include 0.025 wt % to 6 wt % of Pt based on a total weight of the first support. The first support can include at least one of: (i) at least one compound that can include at least one metal having an atomic number of 21, 39, or 57-71 and at least one compound that can include at least one Group 4, 5, 6, 12, 13, 14, 15, or 16 metal or metalloid, and (ii) at least one compound that can include at least one metal having an atomic number of 21, 39, or 57-71 and at least one Group 4, 5, 6, 12, 13, 14, 15, or 16 metal or metalloid. A molar ratio of the at least one metal having the atomic number of 21, 39, or 57-71 to the at least one Group 4, 5, 6, 12, 13, 14, 15, or 16 metal or metalloid is at least 0.03:1. A molar ratio of the at least one metal having the atomic number of 21, 39, or 57-71 to the Pt can be at least 30:1. The second catalyst, if present, can include 0.025 wt % to 50 wt % of Cr based on a total weight of the second support. The second support can include SiO₂, ZrO₂, TiO₂, or a mixture thereof. The process can also include (III) feeding the effluent into a second conversion zone. The process can also include (IV) contacting the effluent with a solid oxygen carrier disposed within the second conversion zone to effect combustion of at least a portion of the molecular hydrogen to produce a conversion product comprising the one or more dehydrogenated hydrocarbons and water.

### BRIEF DESCRIPTION OF THE DRAWINGS

FIG. 1 depicts the yield of C₃H₆ over time produced with a first catalyst that included Pt disposed on a support, according to one or more embodiments described.

FIG. 2 depicts the yield of C₃H₆ over time produced with a second catalyst that included Cr disposed on a support, according to one or more embodiments described.

### DETAILED DESCRIPTION

Various specific embodiments, versions and examples of the invention will now be described, including preferred embodiments and definitions that are adopted herein for purposes of understanding the claimed invention. While the following detailed description gives specific preferred embodiments, those skilled in the art will appreciate that these embodiments are exemplary only, and that the invention may be practiced in other ways. For purposes of determining infringement, the scope of the invention will refer to any one or more of the appended claims, including their equivalents, and elements or limitations that are equivalent to those that are recited. Any reference to the “invention” may refer to one or more, but not necessarily all, of the inventions defined by the claims.

In this disclosure, a process is described as comprising at least one “step.” It should be understood that each step is an action or operation that may be carried out once or multiple times in the process, in a continuous or discontinuous fashion. Unless specified to the contrary or the context clearly indicates otherwise, multiple steps in a process may be conducted sequentially in the order as they are listed, with or without overlapping with one or more other steps, or in any other order, as the case may be. In addition, one or more or even all steps may be conducted simultaneously with regard to the same or different batch of material. For example, in a continuous process, while a first step in a process is being conducted with respect to a raw material just fed into the beginning of the process, a second step may be carried out simultaneously with respect to an intermediate material resulting from treating the raw materials fed into the process at an earlier time in the first step. Preferably, the steps are conducted in the order described.

Unless otherwise indicated, all numbers indicating quantities in this disclosure are to be understood as being modified by the term “about” in all instances. It should also be understood that the precise numerical values used in the specification and claims constitute specific embodiments. Efforts have been made to ensure the accuracy of the data in the examples. However, it should be understood that any measured data inherently contains a certain level of error due to the limitation of the technique and/or equipment used for acquiring the measurement.

Certain embodiments and features are described herein using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated.

The indefinite article “a” or “an”, as used herein, means “at least one” unless specified to the contrary or the context clearly indicates otherwise. Thus, embodiments using “a reactor” or “a conversion zone” include embodiments where one, two or more reactors or conversion zones are used, unless specified to the contrary or the context clearly indicates that only one reactor or conversion zone is used.

The term “hydrocarbon” means (i) any compound consisting of hydrogen and carbon atoms or (ii) any mixture of two or more such compounds in (i). The term “Cn hydrocarbon,” where n is a positive integer, means (i) any hydrocarbon compound comprising carbon atom(s) in its molecule at the total number of n, or (ii) any mixture of two or more such hydrocarbon compounds in (i). Thus, a C2 hydrocarbon can be ethane, ethylene, acetylene, or mixtures of at least two of these compounds at any proportion. A “Cm to Cn hydrocarbon” or “Cm-Cn hydrocarbon,” where m and n are positive integers and m<n, means any of Cm, Cm+1, Cm+2, . . . , Cn−1, Cn hydrocarbons, or any mixtures of two or more thereof. Thus, a “C2 to C3 hydrocarbon” or “C2-C3 hydrocarbon” can be any of ethane, ethylene, acetylene, propane, propene, propyne, propadiene, cyclopropane, and any mixtures of two or more thereof at any proportion between and among the components. A “saturated C2-C3 hydrocarbon” can be ethane, propane, cyclopropane, or any mixture thereof of two or more thereof at any proportion. A “Cn+ hydrocarbon” means (i) any hydrocarbon compound comprising carbon atom(s) in its molecule at the total number of at least n, or (ii) any mixture of two or more such hydrocarbon compounds in (i). A “Cn− hydrocarbon” means (i) any hydrocarbon compound comprising carbon atoms in its molecule at the total number of at most n, or (ii) any mixture of two or more such hydrocarbon compounds in (i). A “Cm hydrocarbon stream” means a hydrocarbon stream consisting essentially of Cm hydrocarbon(s). A “Cm-Cn hydrocarbon stream” means a hydrocarbon stream consisting essentially of Cm-Cn hydrocarbon(s).

For the purposes of this disclosure, the nomenclature of elements is pursuant to the version of the Periodic Table of Elements (under the new notation) as provided in Hawley's Condensed Chemical Dictionary, 16th Ed., John Wiley & Sons, Inc., (2016), Appendix V. For example, a Group 8 element includes Fe, a Group 9 element includes Co, and a group 10 element includes Ni. The term “metalloid”, as used herein, refers to the following elements: B, Si, Ge, As, Sb, Te, and At. In this disclosure, when a given element is indicated as present, it can be present in the elemental state or as any chemical compound thereof, unless it is specified otherwise or clearly indicated otherwise by the context.

The term “alkane” means a saturated hydrocarbon. The term “cyclic alkane” means a saturated hydrocarbon comprising a cyclic carbon ring in the molecular structure thereof. An alkane can be linear, branched, or cyclic.

The term “aromatic” is to be understood in accordance with its art-recognized scope, which includes alkyl substituted and unsubstituted mono- and polynuclear compounds.

The term “rich” when used in phrases such as “X-rich” or “rich in X” means, with respect to an outgoing stream obtained from a device, e.g., a conversion zone, that the stream comprises material X at a concentration higher than in the feed material fed to the same device from which the stream is derived. The term “lean” when used in phrases such as “X-lean” or “lean in X” means, with respect to an outgoing stream obtained from a device, e.g., a conversion zone, that the stream comprises material X at a concentration lower than in the feed material fed to the same device from which the stream is derived.

The term “selectivity” refers to the production (on a carbon mole basis) of a specified compound in a catalytic reaction. As an example, the phrase “an alkane hydrocarbon conversion reaction has a 100% selectivity for an olefin hydrocarbon” means that 100% of the alkane hydrocarbon (carbon mole basis) that is converted in the reaction is converted to the olefin hydrocarbon. When used in connection with a specified reactant, the term “conversion” means the amount of the reactant consumed in the reaction. For example, when the specified reactant is propane, 100% conversion means 100% of the propane is consumed in the reaction. In another example, when the specified reactant is propane, if one mole of propane converts to one mole of methane and one mole of ethylene, the selectivity to methane is 33.3% and the selectivity to ethylene is 66.7%. Yield (carbon mole basis) is conversion times selectivity.

### Overview

In some embodiments, a hydrocarbon-containing feed that includes one or more alkanes, e.g., C₂-C₁₆ linear or branched alkanes and/or C₄-C₁₆ cyclic alkanes, and/or one or more alkyl aromatic hydrocarbons, e.g., C₈-C₁₆ alkyl aromatic hydrocarbons, can be fed into and a conversion zone and can be contacted with a first catalyst that includes Pt disposed on a first support or a second catalyst that includes Cr disposed on a second support to effect dehydrogenation of at least a portion of the hydrocarbon-containing feed to produce an effluent that can include one or more dehydrogenated hydrocarbons and molecular hydrogen. The effluent can be contacted with a solid oxygen carrier in the conversion zone to effect combustion of at least a portion of the molecular hydrogen to produce a conversion product that can include the one or more dehydrogenated hydrocarbons and water.

In other embodiments, the hydrocarbon-containing feed can be contacted with the first catalyst or the second catalyst in a first conversion zone to effect dehydrogenation of at least a portion of the hydrocarbon-containing feed to produce the dehydrogenation effluent that can include the one or more dehydrogenated hydrocarbons and molecular hydrogen. The dehydrogenation effluent can be contacted with a solid oxygen carrier in a second conversion zone to effect combustion of at least a portion of the molecular hydrogen to produce a conversion product that can include the one or more dehydrogenated hydrocarbons and water.

Contacting the effluent with the solid oxygen carrier in the conversion zone or in the second conversion zone can reduce the solid oxygen carrier from a first state to a second state. In some embodiments, feeding the hydrocarbon-containing feed into the conversion zone or feeding the effluent into the second conversion zone can be stopped and an oxidant feed can be fed into the conversion zone or the second conversion zone. The solid oxygen carrier can be reacted with at least a portion of the oxidant feed to oxidize the solid oxygen carrier from the second state to a third state that is greater than the second state. Introduction of the oxidant feed into the conversion zone or the second conversion zone can be stopped, and the hydrocarbon-containing feed can be reintroduced thereto.

### The First Catalyst

The first catalyst can include Pt supported on a first support. In some embodiments, the first catalyst can include 0.025 wt %, 0.1 wt %, 0.2 wt %, 0.5 wt %, or 1 wt % to 2 wt %, 3 wt %, 4 wt %, 5 wt %, or 6 wt % of the Pt based on the total weight of the first support. The first support can be or can include at least one of: (i) at least one compound that includes at least one metal having an atomic number of 21, 39, or 57-71 and at least one compound that includes at least one Group 4, 5, 6, 12, 13, 14, 15, or 16 metal or metalloid, and (ii) at least one compound that includes at least one metal having an atomic number of 21, 39, or 57-71 and at least one Group 4, 5, 6, 12, 13, 14, 15, or 16 metal or metalloid. In some embodiments, the at least one metal having the atomic number of 21, 39, or 57-71 can be or can include, but is not limited to, one or more of: cerium, yttrium, lanthanum, scandium, praseodymium, neodymium, samarium, lutetium, ytterbium, a combination thereof, or a mixture thereof. In some embodiments, the Group 4, 5, 6, 12, 13, 14, 15, or 16 metal or metalloid can be or can include, but is not limited to, one or more of: zirconium, titanium, vanadium, chromium, molybdenum, zinc, aluminum, silicon, antimony, tellurium, a combination thereof, or a mixture thereof.

As noted above, the first catalyst disclosed herein can remain sufficiently active and stable after many dehydrogenation and regeneration cycles. In contrast, it has been discovered that in a comparative catalyst that includes the same components, but having a different molar ratio of the at least one metal having the atomic number of 21, 39, or 57-71 to the at least one Group 4, 5, 6, 12, 13, 14, 15, or 16 metal or metalloid is less than 0.03:1 or exceeds 2.7:1 and/or the molar ratio of the at least one metal having the atomic number of 21, 39, or 57-71 to the Pt of is less than 30:1, the comparative catalyst after 20 dehydrogenation and regeneration cycles is either unstable, is stable but not very active, and/or is unstable and not very active. Without being bound by theory, it is believed that the composition of the first support contributes to the stability of the first catalyst by re-dispersing agglomerated platinum particles during an oxidation step.

The compound that includes the at least one metal having the atomic number of 21, 39, or 57-71, the compound that includes the at least one Group 4, 5, 6, 12, 13, 14, 15, or 16 metal or metalloid, and/or the compound that includes the at least one metal having the atomic number of 21, 39, or 57-71 and the at least one Group 4, 5, 6, 12, 13, 14, 15, or 16 metal or metalloid can be present as an oxide, a phosphate, a halide, a halate, a sulfate, a sulfide, a borate, a nitride, a carbide, an aluminate, an aluminosilicate, a silicate, a carbonate, metaphosphate, a selenide, a tungstate, a molybdate, a chromite, a chromate, a dichromate, or a silicide. In some embodiments, the compound that includes the at least one metal having the atomic number of 21, 39, or 57-71, the compound that includes the at least one Group 4, 5, 6, 12, 13, 14, 15, or 16 metal or metalloid, and/or the compound that includes the at least one metal having the atomic number of 21, 39, or 57-71 and the at least one Group 4, 5, 6, 12, 13, 14, 15, or 16 metal or metalloid can be an oxide. In some embodiments, the compound that includes the at least one metal having the atomic number of 21, 39, or 57-71 and the at least one Group 4, 5, 6, 12, 13, 14, 15, or 16 metal or metalloid can be in a single crystalline phase.

In some embodiments, when the first support includes the compound that includes the at least one metal having the atomic number of 21, 39, or 57-71 and the compound that includes the at least one Group 4, 5, 6, 12, 13, 14, 15, or 16 metal or metalloid, the first support can be or can include, but is not limited to, at least one of: CeO₂, Y₂O₃, La₂O₃, Sc₂O₃, Pr₆O₁₁, and CePO₄ and at least one of: Al₂O₃, SiO₂, ZrO₂, and TiO₂. In some embodiments, when the first support includes the compound that includes the at least one metal having the atomic number of 21, 39, or 57-71 and the at least one Group 4, 5, 6, 12, 13, 14, 15, or 16 metal or metalloid, the first support can be or can include, but is not limited to, at least one of: CeZrO₂, CeAlO₃, BaCeO₃, and CePO₄.

The first support can have a molar ratio of the at least one metal having the atomic number of 21, 39, or 57-71 to the at least one Group 4, 5, 6, 12, 13, 14, 15, or 16 metal or metalloid of 0.03:1, 0.07:1, 0.1:1, 0.15:1, 0.3:1, 0.5:1, 0.7:1, 1:1, or 1.3:1 to 1.5:1, 1.7:1, 2:1, 2.2:1, 2.4:1, 2.5:1, 2.6:1, or 2.7:1. It should be understood that when the first support includes two or more metals having the atomic number of 21, 39, or 57-71 and/or two or more Group 4, 5, 6, 12, 13, 14, 15, or 16 metals and/or metalloids, the molar ratio of the metal having the atomic number of 21, 39, or 57-71 to the Group 4, 5, 6, 12, 13, 14, 15, or 16 metal or metalloid refers to a total amount of all metal(s) having the atomic number of 21, 39, or 57-71 to a total amount of all Group 4, 5, 6, 12, 13, 14, 15, or 16 metals and/or metalloids.

The first catalyst can have a molar ratio of the at least one metal having the atomic number of 21, 39, or 57-71 to Pt of at least 30:1, at least 40:1, at least 60:1, at least 100:1, at least 200:1, at least 400:1, at least 800:1, at least 1,200:1, at least 1,600:1, or at least 2,000:1. In some embodiments, the first catalyst can have a molar ratio of the at least one metal having the atomic number of 21, 39, or 57-71 to Pt of ≥30:1, ≥40:1, ≥60:1, ≥100:1, ≥200:1, ≥400:1, ≥800:1, ≥1,200:1, ≥1,600:1, ≥2,000:1 to ≤2,400:1, ≤2,000:1, ≤1,600:1, ≤1,200:1, ≤800:1, ≤400:1, ≤200:1, ≤100:1, ≤60:1, ≤40:1, or ≤35:1. It should be understood that if the first catalyst includes two or more metals having the atomic number of 21, 39, or 57-71 that the molar ratio of the at least one metal having the atomic number of 21, 39, or 57-71 to Pt refers to a total amount of all metal(s) having the atomic number of 21, 39, 57-71 to Pt.

It should be understood that in some embodiments, one or more elements such as Sn, Ga, Zn, Ge, In, combinations thereof, mixtures thereof, and/or compounds thereof, can be present in the first catalyst and such elements can be referred to as “promoters” for Pt. The promoter, if present, can improve the selectivity/activity/longevity of the first catalyst for a given upgraded hydrocarbon. In some embodiments, the addition of the promoter can improve the propylene selectivity of the first catalyst when the hydrocarbon-containing feed includes propane. The first catalyst can include the promoter in an amount of 0.01 wt %, 0.05 wt %, 0.1 wt %, 0.2 wt %, 0.3 wt %, 0.4 wt %, 0.5 wt %, 0.6 wt %, 0.7 wt %, 0.8 wt %, 0.9 wt %, or 1 wt % to 3 wt %, 5 wt %, 7 wt %, or 10 wt %, based on the weight of the support.

In some embodiments, the first catalyst can also include one or more alkali metal elements disposed thereon. The alkali metal element, if present, can be or can include, but is not limited to, Li, Na, K, Rb, Cs, a combination thereof, or a mixture thereof. In at least some embodiments, the alkali metal element ca be or can include K and/or Cs. The alkali metal element, if present, can improve the selectivity of the first catalyst for a given upgraded hydrocarbon. The first catalyst can include the alkali metal element in an amount 0.01 wt %, 0.1 wt %, 0.2 wt %, 0.3 wt %, 0.4 wt %, 0.5 wt %, 0.6 wt %, 0.7 wt %, 0.8 wt %, 0.9 wt %, or 1 wt % to 2 wt %, 3 wt %, 4 wt %, or 5 wt %, based on the weight of the first support.

The first catalyst can include the first support in the form of particles and/or monolithic structures having the Pt and, if present, additional components such as the promoter and/or alkali metal, disposed thereon, e.g., via a wash coat. The first catalyst can be in the form of beads, spheres, rings, toroidal shapes, irregular shapes, rods, cylinders, flakes, films, cubes, polygonal geometric shapes, sheets, fibers, coils, helices, meshes, sintered porous masses, granules, pellets, tablets, powders, particulates, extrudates, cloth or web form materials, honeycomb matrix monolith, composites (of the first catalyst and the solid oxygen carrier and/or first support material), including in comminuted or crushed forms.

In some embodiments, the primary, non-aggregated particle size of each compound that includes the at least one metal having the atomic number of 21, 39, or 57-71, the compound that includes the at least one Group 4, 5, 6, 12, 13, 14, 15, or 16 metal or metalloid, and/or the compound that includes the at least one metal having the atomic number of 21, 39, or 57-71 and the at least one Group 4, 5, 6, 12, 13, 14, 15, or 16 metal or metalloid can have an average particle size of 0.2 nm, 1 nm, 5 nm, 10 nm, 25 m, 50 nm, 100 nm, 250 nm, 500 nm, 750 nm, 1,000 nm, 1.5 μm, 3 μm, 5 μm, 7 μm, 10 μm to 20 μm, 35 μm, or 50 μm to 65 μm, 80 μm, 90 μm, 100 μm, 150 μm, 200 μm, 300 μm, or 400 μm.

In other embodiments, the compound that includes the at least one metal having the atomic number of 21, 39, or 57-71, the compound that includes the at least one Group 4, 5, 6, 12, 13, 14, 15, or 16 metal or metalloid, and/or the compound that includes the at least one metal having the atomic number of 21, 39, or 57-71 and the at least one Group 4, 5, 6, 12, 13, 14, 15, or 16 metal or metalloid can be extruded or otherwise formed into any desired monolithic structure and the Pt can be disposed thereon. Suitable monolithic structures can be or can include, but are not limited to, structures having a plurality of substantially parallel internal passages such as those in the form of a ceramic honeycomb.

### The Second Catalyst

The second catalyst can include Cr disposed on a second support. In some embodiments, the second catalyst can include 0.025 wt %, 0.05 wt %, 0.1 wt %, 0.5 wt %, 1 wt %, 3 wt %, 5 wt %, 10 wt %, or 15 wt % to 20 wt %, 25 wt %, 30 wt %, 35 wt %, 40 wt %, 45 wt %, or 50 wt % of Cr based on a total weight of the second support. In some embodiments, the Cr can be in the form of Cr₂O₃. In some embodiments, the second support can be or can include, but is not limited to, SiO₂, ZrO₂, TiO₂, or a mixture thereof.

In some embodiments, the second catalyst can also include one or more alkali metal elements disposed thereon. The alkali metal element, if present, can be or can include, but is not limited to, Li, Na, K, Rb, Cs, a combination thereof, or a mixture thereof. In at least some embodiments, the alkali metal element ca be or can include K and/or Cs. The alkali metal element, if present, can improve the selectivity of the second catalyst for a given upgraded hydrocarbon. The second catalyst can include the alkali metal element in an amount 0.01 wt %, 0.1 wt %, 0.2 wt %, 0.3 wt %, 0.4 wt %, 0.5 wt %, 0.6 wt %, 0.7 wt %, 0.8 wt %, 0.9 wt %, or 1 wt % to 2 wt %, 3 wt %, 4 wt %, or 5 wt %, based on the weight of the second support.

In some embodiments, the second support can also include at least one compound that includes at least one Group 5, 6, 12, 13, 15, or 16 metal or metalloid. If the second support includes the at least one compound that includes the at least one Group 5, 6, 12, 13, 15, or 16 metal or metalloid, such compound can be in present as an oxide, a phosphate, a halide, a halate, a sulfate, a sulfide, a borate, a nitride, a carbide, an aluminate, an aluminosilicate, a silicate, a carbonate, metaphosphate, a selenide, a tungstate, a molybdate, a chromite, a chromate, a dichromate, or a silicide.

### Solid Oxygen Carrier

The solid oxygen carrier can be or can include any one or more materials capable of removing molecular hydrogen produced by the dehydrogenation reaction through selective hydrogen combustion (SHC). During the combustion, oxygen from the solid oxygen carrier reacts with the molecular hydrogen from the dehydrogenation to produce water. The solid oxygen carrier can release lattice oxygen during combustion of molecular hydrogen.

In some embodiments, the solid oxygen carrier can be porous materials that have a pore size that is large enough to admit molecular hydrogen, but small enough to exclude the relatively large alkane, alkyl aromatic hydrocarbons, and the dehydrogenated hydrocarbon, e.g., an olefin, molecules. At the start of the dehydrogenation, the solid oxygen carrier is in a state identified as “SOₓC”, indicating that oxygen is available for removal from the solid oxygen carrier. During the dehydrogenation, molecular hydrogen produced by that reaction can enter the pores of the solid oxygen carrier, where the molecular hydrogen can combust with the oxygen available from the solid oxygen carrier. Since molecular hydrogen produced in the dehydrogenation reaction can more readily migrate into the pores of the solid oxygen carrier than can alkanes, alkyl aromatics, and the dehydrogenated hydrocarbon(s), equilibrium of the dehydrogenation reaction shifts toward increased production of the dehydrogenated hydrocarbons and away from hydrogenation of the dehydrogenated hydrocarbons, alkane oxidation, alkyl aromatic oxidation, and oxidation of the dehydrogenated hydrocarbon(s).

Once the oxygen available for combustion in the solid oxygen carrier is depleted, the solid oxygen carrier will be in a reduced state identified conceptually as “SO_{y}C”, where x is a positive number, y is a positive number, and y is <x. The hydrocarbon feed that includes the alkane and/or alkyl aromatic hydrocarbon(s) can be stopped, the solid oxygen carrier can be re-oxidized from SO_{y}C to an oxidized state that is conceptually identified as “SO_{z}C”, where z is a positive number and >y, and the process is repeated. In some embodiments, z can be equal or substantially equal to x. The reduction and oxidation of the SOC are conceptually exemplified by the following equations: H₂+SOₓC→H₂O+SO_{y}C (Reduction); and O₂+2SO_{y}C→2SO_{z}C (Oxidation).

In some embodiments, the solid oxygen carrier can be or can include a metal oxide, for example a transition metal oxide, having a reversible sorptive affinity for oxidant at elevated temperature. In this context, the term “elevated temperature” means a temperature of 400° C. to 1,000° C., and the term “high sorptive capacity” means an oxygen storage capacity of at least 40 millimoles of oxygen per mole of the solid oxygen carrier that contacts the oxygen at a temperature of 800° C. Such materials include those that sportively remove and release oxidant and those that undergo a chemical and/or physical change in the course of reversible oxidant storage. The solid oxygen carrier can be one that stores oxidant in molecular form, e.g., as molecular oxygen, but this is not required. In some embodiments, the solid oxygen carrier can have capacity for storing and releasing oxidant in atomic or ionic form, e.g., as oxygen atoms and/or oxygen ions. In some embodiments, the solid oxygen carrier can enable the bulk separation and purification of oxygen based on ionic transport, in which the solid oxygen carrier is maintained at high temperature to temporarily store oxygen. Oxygen that contacts the surface of the solid oxygen carrier can be decomposed on the surface of the material and incorporated into the crystalline lattice of the material. Storage of the oxygen can be particularly facilitated over the temperature range of 400° C. to 1,000° C.

In some embodiments, when an oxidant contacts the solid oxygen carrier, the oxidant (typically molecular oxygen, but not limited thereto) is adsorbed and dissociated, with charge transfer acting to cause penetrative flux of oxidant into the solid oxygen carrier. A chemical potential driving force can be employed to effect ionic transport of oxidant into the solid oxygen carrier.

The solid oxygen carrier can be of any suitable size, shape and conformation appropriate to oxidant storage and molecular hydrogen combustion. For example, the material can be in a finely divided form, e.g., beads, spheres, rings, toroidal shapes, irregular shapes, rods, cylinders, flakes, films, cubes, polygonal geometric shapes, sheets, fibers, coils, helices, meshes, sintered porous masses, granules, pellets, tablets, powders, particulates, extrudates, cloth or web form materials, honeycomb matrix monolith, composites (of the solid oxygen carrier with hydrocarbon conversion catalyst and/or support material), including in comminuted or crushed forms. The solid oxygen carrier can be mixed with or coated onto a support or substrate. The solid oxygen carrier can be in the form of finely-divided materials as a part of a thermal support or as one or more coatings on a thermal support substrate to provide a material having oxygen-storage functionality. For example, the solid oxygen carrier can be included as a coating onto, a mixture with, or otherwise associated with a substantially inert substrate included in or with the solid oxygen carrier.

In some embodiments, the solid oxygen carrier can be formed by metal-organic chemical vapor deposition (MOCVD) on suitable supports or substrates using appropriate precursors for the respective metal components of the solid oxygen carrier. Use of MOCVD allows relatively close control of stoichiometry and uniformity of coverage to be achieved. MOCVD can be used to deposit films of multicomponent solid oxygen carriers with a compositional reproducibility on the order of 0.1% and a thickness uniformity of better than 5%.

In other embodiments, the solid oxygen carrier can be formed as bulk articles, e.g., particles, by various manufacturing techniques. Such techniques include powder metallurgy, slurry metallurgy (slip casting, tape casting, etc.) and coextrusion. In still other embodiments, the solid oxygen carrier can be formed via a sol gel technique. Such technique can be advantageous when the solid oxygen carrier is deposited on an inert substrate comprising porous silica, alumina, kieselguhr, or the like. Sol gel techniques can be employed to make up a sol of the precursor constituents of the solid oxygen carrier and to spray, dip-coat, soak, roller coat, or otherwise apply the solution to the substrate. The coated substrate containing the precursor material can be subjected to high temperature, e.g., calcined, to produce the desired solid oxygen carrier.

Transition metal oxides can be particularly useful as solid oxygen carriers. In one embodiment, the solid oxygen carrier can be or can include oxides containing at least one metal having an atomic number of 21-30, 39-48, or 57-71. Said another way, suitable transition metals can include an oxide of at least one Group 3-12 metal and/or a lanthanoid metal. In other embodiments, the solid oxygen carriers can be or can include at least one metal-based component composed of one or more elements from Groups 1, 2 and 3; one or more elements from Groups 4-15; and at least one of oxygen and sulfur.

Perovskites and related materials, such as perovskite-like materials and pyrochlores, can also function as solid oxygen carriers. Perovskites are typically oxygen-containing compounds having the crystal structure, ABO₃, with high-temperature O²⁻ vacancies. Such structures can also be denoted by use of the symbol 8, according to the general formula ABO_{3-δ}. The “A”-site cations can be a Group 3 element, a Group 2 element, a Group 1 element and large cations such as Pb²⁺, Bi³⁺. The “B”-site cations can be 3d, 4d, or 5d transition metal cations such as a cation of a metal having an atomic number of 21-30, 39-48, or 72-80. Multiple cation-type occupations are possible. Framework sites “A” and “B” can be dodecahedral and octahedral, respectively, cf., L. G. Tejuca and J. L. Fierro, Properties and Applications of Perovskite-type Oxides, Marcel Dekker, New York, 1993.

Conventional perovskite remains stable and reversible with regard to changes of δ within a certain range. The value δ can be up to 0.25, e.g., δ can be from 0.05 to 0.25 (although higher values have been reported), at elevated temperature and low oxygen partial pressure, i.e., δ is a function of temperature and partial pressure of oxygen. Perovskite stability can be governed by cation radii of lattice metals in various valence states combined into a parameter “t” called “tolerance factor”, cf., Z. Shao, et al., Sep. Purif Technol., 25 (2001) 419-42. A perovskite structure can be formed at t ranges from 0.75-1.

Typically, the perovskite has the general formulas (1) AₓB_{y}O_{3-δ}, (2) AₓA′_{x′}B_{y}B_{y′}O_{3-δ}, and (3) AₓA′_{x′}A″_{x″}B_{y}B′_{y′}B″_{y″}O_{3-δ} and combinations thereof. In these equations, A, A′, and A″ can independently be selected from ions of atoms having atomic number ranging from 57-71, inclusive, a cation of yttrium, ions of Group 1 atoms, ions of Group 2 atoms, and combinations of two or more, where Group 1 and Group 2 refer to the periodic table of elements. B, B′, and B″ are independently selected from: Mn, Cr, Fe, Co, Ni, and Cu. The values of x, x′, x″, y, y′, and y″ are each real numbers ranging from 0 to 1, and x+x′+x″=0.8-1.0; y+y′+y″=1; and δ is from 0.05 to 0.30.

Compounds isostructural with perovskite (“perovskite-like compounds”) are also suitable solid oxygen carriers and can include those having general formulas (4) A₂BO_{4-δ}, (5) A₂B₂O_{5-δ}, (6) AO(ABO_{3-δ})ₙ, (7) AM₂Cu₃O_{7-δ}, (8) Bi₄V₂₍₁₋ₓ₎Me₂ₓO₁₁₋₃ₓ, and (9) A″B″O₃. In these equations, A is independently selected from ions of atoms having atomic numbers ranging from 57-71, inclusive, a cation of yttrium, ions of Group 1 atoms, ions of Group 2 atoms, and combinations of two or more, where Group 1 and Group 2 refer to the periodic table of elements. B is independently selected from d-block transition metal ions. A″ is an ion of Na or Li, and B″ is an ion of W or Mo. M is a metal cation selected from cations of Group 2 atoms of the periodic table of elements. Me is a metal cation selected from cations of Cu, Bi, and Co atoms. The value for x can be from 0.01 to 1.0, n can be from 1 to about 10; and δ can be from 0.05 to about 0.30.

Pyrochlores are also suitable solid oxygen carriers. Suitable pyrochlores can include those having the general formula (10) A₂B₂O_{7-δ}. In this equation, A is independently selected from ions of atoms having atomic numbers ranging from 57-71, inclusive, a cation of yttrium, ions of Group 1 atoms, ions of Group 2 atoms, and combinations of two or more, where Group 1 and Group 2 refer to the periodic table of elements. B is independently selected from d-block transition metal ions; and δ is from 0.05 to 0.30.

Suitable compounds having formula AₓA′_{x′}B_{y}B′_{y′}O_{3-δ} can be or can include, but are not limited to, La_{0.6}Sr_{0.4}Co_{0.8}Fe_{0.2}O_{3-δ}, Sr_{0.9}Ce_{0.1}Fe_{0.8}Co_{0.2}O_{3-δ}, La_{0.2}Sr_{0.8}Co_{0.6}Fe_{0.2}O_{3-δ}, Ba_{0.5}Sr_{0.5}Co_{0.8}Fe_{0.2}O_{3-δ}, Ca_{0.5}Sr_{0.5}Mn_{0.8}Fe_{0.2}O_{3-δ}, Ca_{0.45}Sr_{0.45}Mn_{0.8}Fe_{0.2}O_{3-δ}, La_{0.8}Sr_{0.2}Ni_{0.4}Co_{0.4}Fe_{0.2}O_{3-δ}, La_{0.6}Sr_{0.4}Cr_{0.2}Fe_{0.8}O_{3-δ}, or a mixture thereof. Suitable compounds having formula A₂BO_{4-δ} can be or can include, but are not limited to, La₂CoO_{4-δ}, La₂MnO_{4-δ}, La₂FeO_{4-δ}, Sr₂CuO_{4-δ}, Sr₂MnO_{4-δ}, or a mixture thereof. Suitable compounds having formula A₂B₂O_{5-δ} can be or can include, but are not limited to, La₂Co₂O_{5-δ}, La₂Mn₂O_{5-δ}, Sr₂Cr₂O_{5-δ}, Ce₂Mn₂O_{5-δ}, or a mixture thereof. Suitable compounds having formula AO(ABO_{3-δ})ₙ can be or can include, but are not limited to, LaO(LaCuO_{3-δ})₅, SrO(LaCrO_{3-δ})₆, GdO(SrFeO_{3-δ})₅, CeO(LaNiO_{3-δ})₆, YO(YMnO_{3-δ})ₙ, CeO(CeMnO_{3-δ})ₙ, or a mixture thereof. Suitable compounds having formula AM₂Cu₃O_{7-δ} can be or can include, but are not limited to, KBa₂Cu₃O_{7-δ}, NaBa₂Cu₃O_{7-δ}, LaBa₂Cu₃O_{7-δ}, MgBa₂Cu₃O_{7-δ}, SrBa₂Cu₃O_{7-δ}, Y_{0.5}La_{0.5}BaCaCu₃O_{7-δ}, Y_{0.8}La_{0.2}Ba_{0.8}Sr_{1.2}Cu₃O_{7-δ}, Y_{0.7}La_{0.3}Ba_{0.5}Sr_{1.2}Cu₃O_{7-δ}, Y_{0.9}La_{0.1}Ba_{0.6}Ca_{0.6}Sr_{0.8}Cu₃O_{7-δ}, or a mixture thereof. Suitable compounds having formula Bi₄V₂₍₁₋ₓ₎Me₂ₓO₁₁₋₃ₓ can be or can include, but are not limited to, Bi₄VCuO_{9.5}, Bi₄V_{0.6}Co_{1.4}O_{8.9}, Bi₄V_{1.4}Bi_{0.6}O_{10.1}, Bi₄V_{1.6}Cu_{0.4}O_{10.4}, or a mixture thereof. Suitable compounds having formula A″B″O₃ can be or can include, but are not limited to, LaFeO₃, SrCoO₃, SrFeO₃, or a mixture thereof. Suitable pyrochlores can be or can include, but are not limited to, Mg₂Fe₂O_{7-δ}, Mg₂Co₂O_{7-δ}, Sr₂Mo₂O_{7-δ}, or a mixture thereof. Examples of suitable perovskite, perovskite-like, and pyrochlore compounds can include those disclosed in U.S. Pat. No. 7,338,549.

Solid oxygen carriers can also be or include, but are not limited to, cerium-containing and praseodymium-containing metal oxides, including one or more of CeO₂, Pr₆O₁₁, CeO₂—ZrO₂, CuO—CeO₂, FeOₓ—CeO₂ (1≤x≤1.5), MnOₓ—CeO₂ (1≤x≤3.5), and Pr₆O₁₁—CeO₂. Other suitable solid oxygen carriers can be or can include at least one metal-based component that includes one or more elements from Groups 1, 2 and 3 of the Periodic Table; one or more elements from Groups 4-15; and at least one of oxygen and sulfur. Examples of such solid oxygen carriers can include those described in U.S. Pat. Nos. 7,122,492; 7,122,493; 7,122,495; and 7,125,817.

Other suitable solid oxygen carriers can be or can include 10 wt % or more of at least one first row transition metal (Group 3-12 metal) that has multiple redox states and one or more alkali metal salts that include at least one of an alkali metal oxide and an alkali metal halide, having a molar ratio of the at least one Group 3 metal to the alkali metal in the solid oxygen carrier of 0.5 to 100, and where the solid oxygen carrier has an oxygen storage capacity of 0.5 wt % or more. In some embodiments, the first row transition metal can be or can include Mn, Fe, Co, Ni, Cu, and/or another first row transition metal that has multiple redox states. Suitable solid oxygen carriers having this composition can include those described in WO Publication No. WO2021/025938.

In some embodiments, the solid oxygen carrier can include a metal in oxide form supported on a carrier, where the metal can be or can include, but is not limited to, an alkali metal, an alkaline earth metal, copper, chromium, molybdenum, vanadium, cerium, yttrium, scandium, tungsten, manganese, iron, cobalt, nickel, silver, bismuth, or a combination thereof. In some embodiments, the carrier can be or can include, but is not limited to, aluminum oxides, aluminum hydroxides, aluminum trihydroxide, boehmite, pseudo-boehmite, gibbsite, bayerite, transition aluminas, alpha-alumina, gamma-alumina, silica/alumina, silica, silicates, aluminates, calcium aluminate, barium hexaaluminate, calcined hydrotalcites, zeolites, zinc oxide, chromium oxides, magnesium oxides, zirconia oxides, and a combination thereof.

### Active Material Composites

The arrangement or distribution of the catalyst and the solid oxidant carrier with respect to one another is not critical. In some embodiments, however, it can be beneficial for of the catalyst and the solid oxygen carrier to be located proximate to one another, e.g., as an active material composite. For example, in some embodiments, the solid oxygen carrier can be disposed on a surface of the first catalyst or on a surface of the second catalyst. In other embodiments, however, it can be beneficial for the catalyst and the solid oxygen carrier to be located separate from one another, e.g., in a first and a second conversion zone, respectively. In still other embodiments, it can be beneficial for the catalyst and the solid oxygen carrier to be relatively proximate, but not necessarily intimately combined or mixed as in an active material composite. For example, the catalyst and solid oxygen carrier can be arranged in alternating beds or layers with respect to one another.

In some embodiments, the first catalyst or the second catalyst and the solid oxygen carrier can each be in the form of a plurality of particles, and the first catalyst or the second catalyst and the solid oxygen carrier can be mixed with one another. In other embodiments, the first catalyst or the second catalyst and the solid oxygen carrier can each be in the form of a plurality of particles, and the first catalyst or the second catalyst and the solid oxygen carrier can be arranged in alternating layers. In other embodiments, the first catalyst or the second catalyst and the solid oxygen carrier can each be in the form of a plurality of particles, and the first catalyst or the second catalyst and the solid oxygen carrier can be arranged in staged beds with respect to one another. Suitable active material composites can be prepared via well-known processes such as those disclosed in U.S. Patent Application Publication No. 2016/0318828.

### Process for Dehydrogenating Hydrocarbons

The conversion zone can be located within a reactor, such as a tube reactor. The reactor can include one or more conversion zones disposed therein. In some embodiments, a plurality of reactors can be used. The plurality of reactor can be arranged in series, parallel, or series-parallel. In some embodiments, the conversion zone can include one or more fixed bed reactors containing the same or different catalysts, a moving bed reactor, a fluidized bed reactor, or a combination thereof.

In some embodiments, the conversion zone can be substantially isothermal during the process. In other embodiments, the conversion zone can be non-isothermal during the process. In other embodiments, the conversion zone can be non-isothermal at the start of the process and isothermal conditions can be established during the course of the process. The reactor can be cycled between a dehydrogenation mode and a regeneration mode. The dehydrogenation mode can operate for a first time interval, during which a flow of a first feed, e.g., an alkane-containing feed, can be introduced into the conversion zone. At least a portion of the alkane in the first feed can be dehydrogenated in the presence of a catalytically effective amount of the catalyst disclosed herein. The amount of the Pt and optionally other catalytically active metals can be present in a sufficient amount to provide catalytic dehydrogenation functionality under the specified process conditions. The solids oxygen carrier can be present in a sufficient amount to provide oxidant storage and selective hydrogen combustion functionality under the specified conditions.

During the dehydrogenation mode, the conversion zone can be maintained at a desired dehydrogenation temperature by adding or removing heat from conversion zone components, the feed or components thereof, and/or the effluent or components thereof. In some embodiments, the conversion zone can be heated to a temperature of 400° C., 450° C., or 475° C. to 500° C., 600° C., or 700° C.

At least a portion of the molecular hydrogen in the dehydrogenation product can be combusted in the reaction zone in the presence of an oxidant that is associated with the solid oxygen carrier. Combustion of the molecular hydrogen with the oxidant associated with eh solid oxygen carrier produces water in the conversion product that can be separated from the reaction product, e.g., downstream of the conversion zone, such as by one or more of fractionation, extraction, gravitational settling, etc.

During the dehydrogenation mode, the conversion zone can be maintained or controlled at a pressure effective for carrying out the dehydrogenation and molecular hydrogen combustion reactions. In some embodiments, the pressure within the conversion zone can be ≥0) kPa absolute and ≤3,500 kPa absolute. In some embodiments, the pressure within the conversion zone can be 30 kPa absolute, 70 kPa absolute, 100 kPa absolute, or 125 kPa absolute to 350 kPa absolute, 750 kPa absolute, 1,000 kPa absolute, or 2,500 kPa absolute. The flow of hydrocarbon feed into the conversion zone can be carried out to achieve a weight hourly space velocity (WHSV) effective for carrying out the dehydrogenation process. In some embodiments, the WHSV can be 0.1 hr 1, 0.5 hr⁻¹, 1 hr⁻¹, or 10 hr⁻¹ to 30 hr⁻¹, 50 hr⁻¹, 75 hr⁻¹, or 100 hr⁻¹.

During dehydrogenation mode, the solid oxygen carrier can be reduced from an oxidized state (SOₓC) to a reduced state SO_{y}C, where x and y are positive real numbers and x>y. Dehydrogenation mode can be carried out until (i) alkane and/or alkyl aromatic conversion (indicated by an increase in unreacted alkane in the reaction product) is ≤90% of the conversion at the start of dehydrogenation mode, e.g., ≤85%, or ≤80%; and/or (ii) selectivity for the desired dehydrogenated product such as an olefin (indicated by the amount of desired olefin in the reaction product) is ≤90% of that at the start of dehydrogenation mode, e.g., ≤85%, or ≤80. Typically, when this occurs, the flow of the first feed through the reaction zone can be curtailed or ceased, so that regeneration mode can be carried out. In some embodiments, the first time interval can be ≥1 second, ≥100 seconds, ≥10³ seconds, ≥10⁴ seconds, ≥10⁵ seconds, or ≥10⁶ seconds.

Regeneration mode can include replenishing at least a portion of the oxidant into the solid oxygen carrier that was consumed during the dehydrogenation mode and removing at least a portion of any coke that may have accumulated thereon and/or at least a portion of any coke that may have accumulated on the catalyst. The oxygen “vacancies” in the solid oxygen carrier can be replaced by regenerating the catalyst in the presence of a suitable oxidant, e.g., molecular oxygen, under regeneration conditions. Regeneration can be performed by exposing the solid oxygen carrier to an oxygen-containing feed (such as air) at a regeneration temperature of 400° C. to 1,000° C.

Regeneration mode can be carried out during a second time interval. The second time interval can be a period of time sufficient to (i) replenish ≥50 wt %, ≥75 wt %, or ≥90 wt % of the original oxidant storage capacity of the solid oxygen carrier, and/or (ii) remove ≥50 wt %, ≥75 wt %, or ≥90 wt % of any accumulated coke disposed on the catalyst. In some embodiments, the duration of the regeneration mode can be ≤50%, ≤25%, or ≤10% of the duration of dehydrogenation mode. In some embodiments, the duration of the regeneration mode can be ≤5×10⁴ seconds, ≤1×10³ seconds, ≤100 seconds, or ≤10 seconds.

The solid oxygen carrier can be oxidized (or re-oxidized) during regeneration mode from state SO_{y}C to state SO_{z}C, where z is a positive real number and z>y. Once oxidized to state SO_{z}C, the solid oxygen carrier can again function as a source of oxidant for the selective combustion of molecular hydrogen, which can also be carried out in the conversion zone. Although z can have substantially the same value as x, this is not required.

In an alternative embodiment, regeneration mode is not carried out. In such case, spent catalyst and/or the solid oxygen carrier can be removed from the conversion zone and replaced with fresh or regenerated material. Replacement can be carried out continuously, e.g., utilizing conventional fluidized catalyst or slurry catalyst technology, in a batch method, and/or in combinations thereof.

In another embodiment, a first conversion zone can include the catalyst disposed therein and a second conversion zone can include the solid oxygen carrier disposed therein. The effluent produced in the first conversion zone can be introduced into the second conversion zone where the solid oxygen carrier can selectively combust at least a portion of the molecular hydrogen.

In some embodiments, the conversion zone can be substantially isothermal during the second time interval, but this is not required. In some embodiments, at least a portion of the oxidant, e.g., molecular oxygen, from the second feed can be stored by the solid oxygen carrier upon completion of the regeneration mode. In some embodiments, at least a portion of the oxidant in the second feed can be used to remove coke deposits from the catalyst and/or the solid oxygen carrier, which can substantially restore the dehydrogenation activity of the catalyst. Once sufficient re-oxidation has occurred, i.e., sufficient oxidant is stored to carry out the molecular hydrogen combustion during dehydrogenation mode, the flow of the second feed through the reaction zone can be curtailed or ceased, and the flow of the hydrocarbon feed can be re-established.

In some embodiments, the oxidant content in the second feed, e.g., molecular oxygen content, can be ≥1 mol %, such as 10 mol % to 35 mol %. Doing so provides excess oxidant over that needed for replenishing the oxidant in the solid oxygen carrier. The excess oxidant can increase the rate of coke removal from the catalyst and/or solid oxygen carrier, so that the time needed for coke removal and the time needed for oxidant replacement can be sufficiently similar, which can reduce the duration of the second time interval and lead to an increased yield in the dehydrogenated product produced within a given period of time. In some embodiments, the regeneration mode can be carried out for a sufficient amount of time to (i) replenish ≥50 wt %, ≥75 wt %, or ≥90 wt % of the original oxidant storage capacity of the solid oxygen carrier and/or (ii) remove ≥50 wt % of, ≥75 wt %, or ≥90 wt % of accumulated coke on the catalyst and/or solid oxygen carrier.

In some embodiments, the regeneration mode can also include introducing a reducing feed that can include molecular hydrogen, carbon monoxide, steam, or a mixture thereof into the conversion zone.

Alternating flows of first and second feeds, e.g., alternating first and second time intervals, can be repeated continuously or semi-continuously. One or more additional feeds, e.g., one or more sweep fluids, can be utilized between flows of the first and second feeds, e.g., to remove undesired material from the reactors, such as non-combustible particulates including soot. The additional feed(s) can be inert under conditions specified for the first and second time intervals.

The first and second feeds can be contacted with the catalyst and the solid oxygen carrier in one or more of an upward, downward, or radial flow fashion. The first and second feeds and conversion product removed from the conversion zone can be in the liquid phase, mixed liquid and vapor phase, or in the vapor phase. In some embodiments, a fixed bed reactor can be employed, e.g., one having a plurality of beds of one or more of the catalyst and solid oxygen carrier. When the conversion zone includes a plurality of beds, each bed can be of the same composition or different.

Appropriate thermal regulation of the selective hydrogen combustion reaction can be carried out through the use of one or more solid oxygen carriers and/or by distributing the solid oxygen carrier(s) in the conversion zone to achieve a desired temperature profile. Additional regulation of the heat of reaction, if needed, can be carried out through thermal moderation of the temperature profile in the conversion zone. Establishing and maintaining a desired temperature profile can be carried out by (i) selecting an appropriate type and amount of solid oxygen carrier, (ii) appropriate distribution of the solid oxygen carrier within the conversion zone, and/or (iii) utilizing additional thermal moderation of the conversion zone to achieve a desired temperature profile.

In some embodiments, the type and amount of the solid oxygen carrier can be selected to provide sufficient desorbed oxidant for combustion of the molecular hydrogen under the given dehydrogenation conditions, but little if any additional oxidant beyond what is needed for the combustion of the molecular hydrogen. Since adsorption is typically exothermic and desorption is typically endothermic, desorption of additional oxidant would undesirably compete with the endothermic dehydrogenation reaction for heat produced by the combustion. Consequently, it can also be desirable for the solid oxygen carrier to be one having a heat of desorption that is less than the heat needed for the dehydrogenation of the hydrocarbon feed.

In some embodiments, solid oxygen carriers that include perovskite, material isostructural with perovskite, pyrochlore, and/or material isostructural with pyrochlore can be particularly suitable, especially such of those having a pore size appropriate for selective molecular hydrogen combustion. Additionally, in some embodiments, since the temperature within the conversion zone can more easily be maintained at a desired temperature when desorbed oxidant combustion occurs proximate to the dehydrogenation sites of the catalyst, it can be desirable for the catalyst to include the solid oxygen carrier impregnated into and/or onto the catalyst and/or vice versa. In some embodiments, the impregnation of the solid oxygen carrier onto the catalyst or vice versa can be carried out to provide a substantially-uniform concentration about the catalyst or solid oxygen carrier.

Additional thermal moderation of the temperature within the conversion zone, if needed, can be carried out by removing heat from or adding heat to one or more locations in the conversion zone. In some embodiments, external and/or internal transfers of heat to and/or from one or more beds disposed within the conversion zone can be used. Examples of reactors that can be configured to do this can include radial flow catalyst bed reactors having heat transfer tubes arranged within the beds. The heat transfer tubes can be arranged in a variety of configurations including vertical, horizontal, and/or helical tubular arrangements. In some embodiments, the conversion zone can include at least one bed of material, e.g., catalyst, the solid oxygen carrier, or both, and a plurality of helical tubes within bed. During dehydrogenation mode, a heat transfer fluid can be conveyed through the tubes, the temperature of the heat transfer fluid being regulated to supply or remove heat in order to maintain the bed in at a desired isothermal profile. The bed can be arranged axially and/or radially within the reaction zone, in proximity to the heat transfer tubes. In some embodiments, reactors having suitable conversion zones can include those described in German Patent Application No. DE-A-3 318 098, and in U.S. Pat. Nos. 4,339,413 and 4,636,365. In some embodiments, preferably at least 50% of the heat transfer tubes in the conversion zone can be configured so that they are each exposed to substantially the same thermal load during dehydrogenation mode. For example, when additional heat is removed from the conversion zone by a heat transfer fluid comprising liquid water, at least 50% of the tubes produce the same amount of steam (uniform distribution of the water and steam inside the tubes). Such conversion zones can include those disclosed in U.S. Pat. No. 6,958,135. In some embodiments, ≥25 mol %, ≥50 mol %, ≥75 mol %, or ≥90 mol % of the molecular hydrogen in the effluent can be consumed during dehydrogenation mode using oxygen released from the solid oxygen carrier.

Alternatively or in addition to the use of heat transfer tubes, the first feed can be heated before or during its introduction into the conversion zone to provide additional thermal moderation. The preheating can be applied throughout dehydrogenation mode, or more typically, for an initial period at the start of the first interval in order to initiate dehydrogenation of the hydrocarbon feed. After this initial period, heat released in the conversion zone by combustion of the molecular hydrogen produced during the dehydrogenation can substitute for at least a portion of the first feed preheating for maintaining the established isothermal temperature profile for the remainder of dehydrogenation mode.

The dehydrogenation process disclosed herein can provide superior overall dehydrogenation properties including one or more of high selectivity to a desired dehydrogenated product, e.g., an olefin, high conversion of the hydrocarbon, e.g., an alkane, and a low deactivation rate of the catalyst compared to conventional processes. Selectivity to the desired olefin can be, e.g., ≥60%, ≥70%, ≥80%, ≥90%, or ≥95% on a molar basis. In particular, when the hydrocarbon feed includes propane, the process has high selectivity to propylene. In some embodiments, alkane conversion, e.g., propane conversion, can be ≥10%, ≥20%, ≥40%, ≥60%, or ≥80%.

Olefin compounds produced by the process can be cyclic or acyclic, meaning that double bond of the olefin can be located between carbon atoms forming part of a cyclic (closed-ring) or part of an open-chain grouping, although typically the olefin is acyclic. The olefin can have more than one double bond, although typically the olefin has one double bond only. The process is particularly effective in converting a lower alkane, e.g., a particular C₃-C₅ alkane, to respective lower olefin of the same carbon number, e.g., a particular C₃-C₅ olefin.

### Feed Compositions

The process utilizes a first feed or hydrocarbon feed that can include the alkane and/or alkyl aromatic hydrocarbon(s) during dehydrogenation mode and optionally a second feed that can include an oxidant during an optional regeneration mode.

The alkane, when present in the hydrocarbon feed, can be an alkane compound having n carbon atoms, with n being an integer ≥2. The alkane hydrocarbon and the olefin hydrocarbon produced therefrom can be of the same order, i.e., have the same value of n. In some embodiments, the alkane can be selected from among C₂ to C₂₀ alkanes, C₂ to C₁₈ alkanes, C₃ to C₁₂ alkanes, or C₃ to C₅ alkanes. In some embodiments, the alkane can be or can include, but is not limited to, ethane, propane, n-butane, isobutane, n-pentane, isopentane, n-hexane, 2-methylpentane, 3-methylpentane, 2,2-dimethylbutane, n-heptane, 2-methylhexane, 2,2,3-trimethylbutane, cyclopentane, cyclohexane, methylcyclopentane, ethylcyclopentane, n-propylcyclopentane, 1,3-dimethylcyclohexane, or a mixture thereof. For example, the hydrocarbon feed can include propane, which can be dehydrogenated to produce propylene, and/or isobutane, which can be dehydrogenated to produce isobutylene. In some embodiments, substantially all of the alkane in the hydrocarbon feed can be a single or a “designated” alkane such as propane. In some embodiments, the first feed can include ≥50 mol %, ≥75 mol %, ≥95 mol %, ≥98 mol %, or ≥99 mol % of propane based on a total weight of all hydrocarbons in the hydrocarbon feed.

In addition to the designated alkane, the hydrocarbon feed can also include one or more additional hydrocarbons, e.g., additional alkanes. When the hydrocarbon feed includes a designated alkane and additional hydrocarbon, the hydrocarbon feed can include ≥50 wt %, ≥60 wt %, ≥70 wt %, ≥80 wt %, ≥90 wt %, ≥95 wt %, or ≥99 wt % of the designated alkane based on the total weight of hydrocarbons in the hydrocarbon feed.

The alkyl aromatic, when present in the hydrocarbon feed, can include any one or more alkyl aromatic hydrocarbons. In some embodiments, the alkyl aromatic hydrocarbon can be selected from among C₇ to C₂₀ alkyl aromatics, C₇ to C₁₈ alkyl aromatics, C₇ to C₁₂ alkyl aromatics, or C₇ to C₁₀ alkyl aromatics. In some embodiments, the alkyl aromatic can be or can include one or more ethyl substituted benzenes, one or more ethyl toluenes, or a mixture thereof. In some embodiments, the ethyl substituted benzene can be or can include, but is not limited to, ethyl benzene, which can be dehydrogenated to produce styrene. In some embodiments, the ethyl toluene can be or can include, but is not limited to, para-ethyltoluene, which can be dehydrogenated to produce para-methylstyrene.

In some embodiments, the first feed can be diluted, e.g., with one or more diluents such as one or more substantially-inert materials. For example, the first feed can be diluted with essentially inert fluid, such as molecular nitrogen. Substantially inert in this context means that ≤0.1 wt % of the material present in the hydrocarbon feed reacts with an alkane, an alkyl aromatic, molecular hydrogen, and/or the dehydrogenated hydrocarbon(s) under the dehydrogenation reaction conditions. In some embodiments, the hydrocarbon feed can include 1 wt %, 5 wt %, 10 wt % to 20 wt %, 30 wt %, or 40 wt % of the diluent based on a total weight of the hydrocarbon feed.

The hydrocarbon feed can be substantially free or free of molecular oxygen. In some embodiments, the hydrocarbon feed can include ≤5 mol %, ≤3 mol %, or ≤1 mol % of molecular oxygen (O₂). It is believed that providing a hydrocarbon feed substantially-free of molecular oxygen substantially prevents oxidative coupling reactions that would otherwise consume at least a portion of the alkane and/or the alkyl aromatic in the hydrocarbon feed.

The oxidant-containing feed or regenerating feed can include one or more oxidants and optionally one or more diluents. The oxidant can be or can include, but is not limited to, molecular oxygen, ozone, and gases that yield oxygen such as N₂O. Oxidants that can be liquid or solid at ambient conditions can also be used provided that such oxidants can be introduced into the conversion zone. The oxidant-containing feed can include sufficient oxidant for storage within the solid oxygen carrier. For example, the oxidant-containing feed can include ≥0.1 mol %, ≥0.5 mol %, ≥5 mol %, ≥10 mol, 20 mol %, ≥25 mol %, or ≥30 mol % of the oxidant based on a total amount of the oxygen-containing feed. For example, the amount of oxidant in the oxygen-containing feed can be about 0.1 mol %, about 0.3 mol %, or about 0.5 mol % to 1 mol %, 25 mol %, or 99.9 mol %. The remainder of the oxidant-containing feed or at least a portion thereof can be a diluent, e.g., a material that is substantially unreactive (or only mildly so) with the oxidant under the conditions utilized for replenishing the oxygen in the solid oxygen carrier.

In some embodiments, the oxidant-containing feed can include molecular oxygen. For example, the oxidant-containing feed can include ≥90 mol %, ≥95 mol %, ≥98 mol %, ≥99 mol %, or ≥99.5 mol % of molecular oxygen based on a total amount of oxidant in the oxygen-containing feed. The molecular oxygen can be molecular oxygen in air or molecular oxygen obtained or derived from air, e.g., by separation. Molecular nitrogen obtained or derived from air can be utilized as the diluent. In some embodiments, the oxidant can include molecular oxygen in air, and the diluent includes molecular nitrogen in air. For example, the second feed can be or can include air.

The reducing feed, if used, can be or can include, but is not limited to, molecular hydrogen, carbon monoxide, steam, or a mixture thereof. In some embodiments, the reducing feed can include a diluent such as molecular nitrogen. The reducing feed can be introduced after the oxidant-containing feed and before the hydrocarbon feed is reintroduced to reduce the active metal (Pt) in the catalyst. The reducing feed can also react with any residual oxidant, e.g., molecular oxygen, to remove at least a portion of the residual oxidant from the conversion zone.

### Recovery and Use of the Conversion Product

The conversion product can include at least one desired olefin and/or at least one desired dehydrogenated alkyl aromatic, water, unreacted hydrocarbon, other unreacted first feed components, unreacted molecular hydrogen, etc. When present, the amount of unreacted alkane can be low, e.g., ≤10 mol %, ≤5 mol %, or ≤1 mol %. When present, the amount of unreacted molecular hydrogen can also be low, e.g., ≤1 mol % or ≤0.1%. Olefin can be removed from the reaction product by any convenient process, e.g., by cone or more conventional processes. One such process can include cooling the conversion product to condense at least a portion of any water and any heavy hydrocarbon that may be present, leaving the olefin and any unreacted alkane or alkyl aromatic primarily in the vapor phase. Olefin and unreacted alkane or alkyl aromatic can then be removed from the reaction product in one or more separator devices. For example, one or more splitters and/or distillation columns can be used to separate the dehydrogenated product from the unreacted hydrocarbon feed.

In some embodiments, a recovered olefin, e.g., propylene, can be used for producing polymer, e.g., recovered propylene can be polymerized to produce polymer having segments or units derived from the recovered propylene such as polypropylene, ethylene-propylene copolymer, etc. Recovered isobutene can be used, e.g., for producing one or more of: an oxygenate such as methyl tert-butyl ether, fuel additives such as diisobutene, synthetic elastomeric polymer such as butyl rubber, etc.

### EXAMPLES

The foregoing discussion can be further described with reference to the following non-limiting examples.

### Example 1

In this example, the catalyst was PtSn/CeO₂/Al₂O₃ and the solid oxygen carrier was Fe₂O₃/Li₂O/K₂O. The catalyst included 0.8 wt % of Pt, 2.4 wt % of SnO₂, and 30 wt % of CeO₂. The molar ratio of Fe:Li:K was 3:0.75:0.75. The catalyst and the solid oxygen carrier were packed in a quartz reactor tube in a stacked bed fashion as follows: 0.15 g of the catalyst/0.5 g of the solid oxygen carrier/0.15 g of the catalyst/0.5 g of the solid oxygen carrier/0.15 g of the catalyst/0.5 g of the solid oxygen carrier/0.15 g of the catalyst. Quartz wool was used as a physical barrier between each two adjacent layers. A small amount of SiC was used as a diluent in the catalyst layers. The temperature within the reactor tube was 540° C. and the pressure within the reactor tube was at ambient pressure. The feed contained 90 vol % of C₃H₈ and 10 vol % of Ar.

The following process steps were performed.
1. The reactor was heated to a temperature of 540° C. under a helium (He) atmosphere.
2. He was passed through the reactor at a rate of 10 sccm for 10 min.
3. The feed (C₃H₈/Ar) was passed through a reactor by-pass at a rate of 11 sccm for 10 min to establish a gas chromatograph baseline and to reduce transient response. During this time period the catalyst and solid oxygen carrier were under a He atmosphere.
4. The feed was introduced into and passed through the reactor at a rate of 11 sccm for 10 min.
5. He was introduced into the reactor to purge the reactor at a rate of 10 sccm for 10 min.
6. An oxidant (10% O₂ in He) was introduced into the reactor by-pass at a rate of 10 sccm for 10 min to establish a gas chromatography baseline and to reduce transient response. During this time period the catalyst and solid oxygen carrier were under a He atmosphere.
7. The oxidant (10% O₂ in He) was introduced into and passed through the reactor at a rate of 2 sccm for 10 min to 30 min, 5 sccm for 10 min to 30 min, and 10 sccm for 10 min to 60 min.
8. Returned to Step 2.

It should be understood that an optional step that includes introducing H₂ and/or CO and/or other reducing gas can be introduced between step 1 and step 2. A brief introduction of a reducing gas can improve the selectivity/activity of the catalyst without excessive reduction of the SOC.

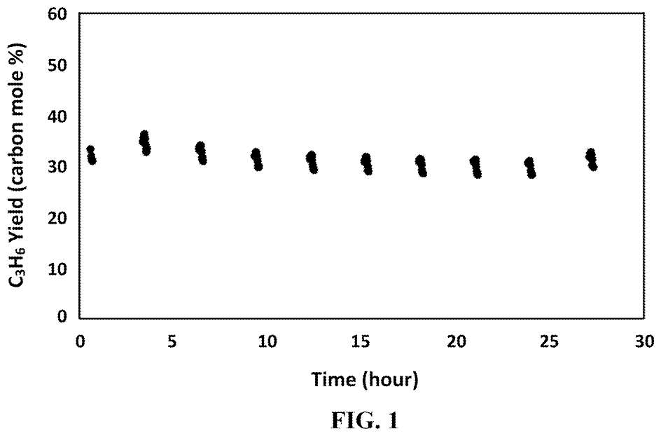

FIG. 1 shows the yield of C₃H₆. In FIG. 1, the x-axis is time in h, the y-axis is yield of C₃H₆ (carbon mole %). The equilibrium yield of propane dehydrogenation without H₂ removal at the testing condition is ˜28.6%. Combining the catalyst and the solid oxygen carrier in the stacked bed arrangement within the reactor clearly enhanced the yield of C₃H₆ above 28.6%. In the last cycle, a brief H₂ reduction for 60 seconds using 10 sccm of 10% H₂ and 90 Ar was introduced between step 2 and step 3 (before introducing the C₃H₈/Ar gas). Some improvement in C₃H₆ yield was observed.

### Example 2

In this example, the catalyst was Cr₂O₃ impregnated on Davisil 923, a commercial silica. The solid oxygen carrier was Fe₂O₃/Li₂O/K₂O. The catalyst included 2.5 wt % of the Cr₂O₃. The molar ratio of Fe:Li:K was 3:0.75:0.75. The Cr₂O₃ catalyst (0.75 g) and a small amount of SiC was a diluent were loaded into the reactor without any solid oxygen carrier and tested to establish a base line in C₃H₆ yield. Then the Cr₂O₃ catalyst (0.75 g) was mixed with 1.5 g of the solid oxygen carrier and a small amount of SiC as the diluent that was loaded into the reactor. The temperature and pressure of the reaction was 540° C. and ambient pressure, respectively. The feed was 12 sccm of 90%/10% C₃H₈/Ar.

The following process steps were performed.
1. The reactor was heated to a temperature of 540° C. under a He atmosphere.
2. He was passed through the reactor at a rate of 10 sccm for 10 minutes.
3. The feed (C₃H₈/Ar) was passed through a reactor by-pass at a rate of 12 sccm for 10 min to establish a gas chromatograph baseline and to reduce transient response. During this time period the catalyst and solid oxygen carrier were under a He atmosphere.
4. The feed was introduced into and passed through the reactor at a rate of 12 sccm for 25 min.
5. He was introduced into the reactor to purge the reactor at a rate of 10 sccm for 10 min.

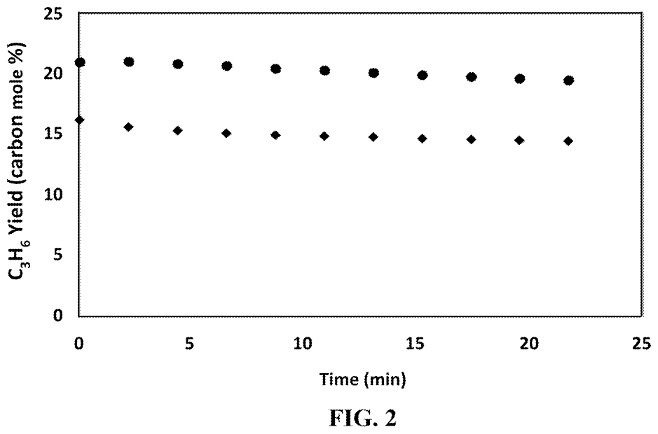

FIG. 2 shows the yield of C₃H₆ alone (♦) and the yield of C₃H₆ mixed with the solid oxygen carrier (●). In FIG. 2, the x-axis is time in min. The y-axis is the C₃H₆ yield. As shown in FIG. 2, an increase in C₃H₆ yield was observed when mixed with the solid oxygen carrier.

### Listing of Embodiments

This disclosure may further include the following non-limiting embodiments.

A1. A process for dehydrogenating a hydrocarbon, comprising: (I) feeding a hydrocarbon-containing feed comprising one or more of C₂-C₁₆ linear or branched alkanes, one or more of C₄-C₁₆ cyclic alkanes, one or more of C₈-C₁₆ alkyl aromatics, or a mixture thereof into a conversion zone; (II) contacting the hydrocarbon-containing feed with a first catalyst comprising Pt disposed on a first support or a second catalyst comprising Cr disposed on a second support within the conversion zone to effect dehydrogenation of at least a portion of the hydrocarbon-containing feed to produce an effluent comprising one or more dehydrogenated hydrocarbons and molecular hydrogen, (i) wherein: the first catalyst comprises 0.025 wt % to 6 wt % of Pt based on a total weight of the first support, the first support comprises at least one of: (i) at least one compound comprising at least one metal having an atomic number of 21, 39, or 57-71 and at least one compound comprising at least one Group 4, 5, 6, 12, 13, 14, 15, or 16 metal or metalloid, and (ii) at least one compound comprising at least one metal having an atomic number of 21, 39, or 57-71 and at least one Group 4, 5, 6, 12, 13, 14, 15, or 16 metal or metalloid, a molar ratio of the at least one metal having the atomic number of 21, 39, or 57-71 to the at least one Group 4, 5, 6, 12, 13, 14, 15, or 16 metal or metalloid is at least 0.03:1, and a molar ratio of the at least one metal having the atomic number of 21, 39, or 57-71 to the Pt is at least 30:1, or (ii) wherein: the second catalyst comprises 0.025 wt % to 50 wt % of Cr based on a total weight of the second support, and the second support comprises SiO₂, ZrO₂, TiO₂, or a mixture thereof; and (III) contacting the effluent with a solid oxygen carrier disposed within the conversion zone to effect combustion of at least a portion of the molecular hydrogen to produce a conversion product comprising the one or more dehydrogenated hydrocarbons and water.

A2. The process of A1, wherein the first catalyst is present, and wherein the first catalyst further comprises an alkali metal element disposed on the first support.

A3. The process of A2, wherein the alkali metal element comprises Li, Na, K Rb, Cs, a combination thereof, or a mixture thereof.

A4. The process of A2 or A3, wherein the first catalyst comprises up to 5 wt % of the alkali metal based on the total weight of the first support.

A5. The process of any of A1 to A4, wherein the first catalyst is present, and wherein the molar ratio of the at least one metal having the atomic number of 21, 39, or 57-71 to the at least one Group 4, 5, 6, 12, 13, 14, 15, or 16 metal or metalloid is at least 0.03:1 to 2.7:1.

A6. The process of any of A1 to A5, wherein the first catalyst is present, and wherein the molar ratio of the at least one metal having the atomic number of 21, 39, or 57-71 to the Pt is at least 30 to 5000.

A7. The process of any of A1 to A6, wherein the first catalyst is present, and wherein the at least one compound comprising the at least one metal having the atomic number of 21, 39, or 57-71 or the at least one compound comprising the at least one metal having the atomic number of 21, 39, or 57-71 and the at least one Group 4, 5, 6, 12, 13, 14, 15, or 16 metal or metalloid is an oxide, a phosphate, a halide, a halate, a sulfate, a sulfide, a borate, a nitride, a carbide, an aluminate, an aluminosilicate, a silicate, a carbonate, metaphosphate, a selenide, a tungstate, a molybdate, a chromite, a chromate, a dichromate, or a silicide.

A8. The process of any of A1 to A7, wherein the first catalyst is present, and wherein the at least one metal having the atomic number of 21, 39, or 57-71 comprises at least one of Ce, Y, La, Sc, and Pr.

A9. The process of any of A1 to A8, wherein the first catalyst is present, and wherein the at least one Group 4, 5, 6, 12, 13, 14, 15, or 16 metal or metalloid comprises at least one of Zr, Al, Ti, and Si.

A10. The process of any of A1 to A9, wherein the first catalyst is present, and wherein the first support comprises a mixture of at least one compound comprising CeO₂, Y₂O₃, La₂O₃, Sc₂O₃, Pr₆O₁₁, and CePO₄, and at least one compound comprising Al₂O₃, SiO₂, ZrO₂, and TiO₂.

A11. The process of any of A1 to A10, wherein first catalyst is present, and wherein the first support comprises CeZrO₂, CeAlO₃, BaCeO₃, CePO₄, or a mixture thereof.

A12. The process of A1, wherein the second catalyst is present, and wherein the second catalyst further comprises an alkali metal element disposed on the second support.

A13. The process of A12, wherein the alkali metal element comprises Li, Na, K, Rb, Cs, a compound thereof, or a mixture thereof.

A14. The process of any of A1, A12, or A13, wherein the second catalyst is present, and wherein the second support further comprises at least one compound comprising at least one Group 5, 6, 12, 13, 15, or 16 metal or metalloid.

A15. The process of A14, wherein the at least one compound comprising the at least one Group 5, 6, 12, 13, 15, or 16 metal or metalloid is an oxide, a phosphate, a halide, a halate, a sulfate, a sulfide, a borate, a nitride, a carbide, an aluminate, an aluminosilicate, a silicate, a carbonate, metaphosphate, a selenide, a tungstate, a molybdate, a chromite, a chromate, a dichromate, or a silicide.

A16. The process of any of A1 to A15, wherein the solid oxygen carrier releases lattice oxygen during combustion of the molecular hydrogen.

A17. The process of any of A1 to A16, wherein the solid oxygen carrier is reduced from a first state SOₓC to a second state SO_{y}C during step (III), wherein x is a positive number, y is a positive number, and y is <x, the process further comprising: (IV) stopping feeding of the hydrocarbon-containing feed into the conversion zone; (V) feeding an oxidant feed into the conversion zone; (VI) reacting the solid oxygen carrier with a first portion of the oxidant to oxidize the solid oxygen carrier from the second state to a third state SO_{z}C, wherein z is a positive number, and wherein z is >y; (VII) stopping feeding of the oxidant into the conversion zone; and (VIII) repeating steps (I) to (III).

A18. The process of A17, wherein the process further comprises, after step (VII) and before step (VIII), the following steps: (VIIb) feeding a reducing gas comprising molecular hydrogen, carbon monoxide, steam, or a mixture thereof into the conversion zone; and (VIIc) contacting the catalyst with the reducing gas to reduce at least a portion of the Pt from an oxidized state to a metallic state.

A19. The process of A17 or A18, wherein in step (II), coke is formed on the surface of the catalyst, and wherein in step (VI), a second portion of the oxidant combusts at least a portion of the coke on the surface of the catalyst.

A20. The process of any of A1 to A19, wherein the hydrocarbon-containing feed comprises a C₂ to C₁₈ alkane.

A21. The process of any of A1 to A20, wherein the hydrocarbon-containing feed comprises ethane, propane, butane, pentane, hexane, heptane, octane or a mixture thereof.

A22. The process of any of A1 to A21, wherein the hydrocarbon-containing feed comprises ethylbenzene, ethyl toluene, isopropyl benzene, diethylbenzene, or a mixture thereof.

A23. The process of any of A1 to A22, wherein the hydrocarbon-containing feed contacts the first catalyst or the second catalyst in the conversion zone at a weight hour space velocity of 0.01 hr⁻¹ to 300 hr⁻¹, at a temperature of 300° C. to 750° C., and under an absolute pressure of 10 kPa to 1,000 kPa.

A24. The process of any of A1 to A23, wherein the effluent contacts the solid oxygen carrier in the conversion zone at a weight hour space velocity of 0.01 hr⁻¹ to 300 hr⁻¹, at a temperature of 300° C. to 750° C., and under an absolute pressure of 10 kPa to 1,000 kPa.

A25. The process of any of A1 to A24, wherein the first catalyst or the second catalyst and the solid oxygen carrier are each in the form of a plurality of particles, and wherein the first catalyst or the second catalyst and the solid oxygen carrier are mixed with one another within the conversion zone.

A26. The process of any of A1 to A25, wherein the first catalyst or the second catalyst and the solid oxygen carrier are each in the form of a plurality of particles, and wherein the first catalyst or the second catalyst and the solid oxygen carrier are arranged in alternating layers within the conversion zone.

A27. The process of any of A1 to A26, wherein the first catalyst or the second catalyst and the solid oxygen carrier are each in the form of a plurality of particles, and wherein the first catalyst or the second catalyst and the solid oxygen carrier are arranged in staged beds with respect to one another within the conversion zone.

A28. The process of any of A1 to A27, wherein the solid oxygen carrier comprises a metal in oxide form supported on a carrier, wherein the metal is selected from the group consisting of: an alkali metal, an alkaline earth metal, copper, chromium, molybdenum, vanadium, cerium, yttrium, scandium, tungsten, manganese, iron, cobalt, nickel, silver, bismuth, and a combination thereof.

A29. The process of A28, wherein the carrier is selected from the group consisting of: aluminum oxides, aluminum hydroxides, aluminum trihydroxide, boehmite, pseudo-boehmite, gibbsite, bayerite, transition aluminas, alpha-alumina, gamma-alumina, silica/alumina, silica, silicates, aluminates, calcium aluminate, barium hexaaluminate, calcined hydrotalcites, zeolites, zinc oxide, chromium oxides, magnesium oxides, zirconia oxides, and a combination thereof.

A30. The process of any of A1 to A29 wherein the solid oxygen carrier is disposed on a surface of the first catalyst or a surface of the second catalyst.

B1. A process for dehydrogenating a hydrocarbon, comprising: (I) feeding a hydrocarbon-containing feed comprising one or more of C₂-C₁₆ linear or branched alkanes, one or more of C₄-C₁₆ cyclic alkanes, one or more of C₈-C₁₆ alkyl aromatics, or a mixture thereof into a first conversion zone; (II) contacting the hydrocarbon-containing feed with a first catalyst comprising Pt disposed on a first support or a second catalyst comprising Cr disposed on a second support within the first conversion zone to effect dehydrogenation of at least a portion of the hydrocarbon-containing feed to produce an effluent comprising one or more dehydrogenated hydrocarbons and molecular hydrogen, (i) wherein: the first catalyst comprises 0.025 wt % to 6 wt % of Pt based on a total weight of the first support, the first support comprises at least one of: (i) at least one compound comprising at least one metal having an atomic number of 21, 39, or 57-71 and at least one compound comprising at least one Group 4, 5, 6, 12, 13, 14, 15, or 16 metal or metalloid, and (ii) at least one compound comprising at least one metal having an atomic number of 21, 39, or 57-71 and at least one Group 4, 5, 6, 12, 13, 14, 15, or 16 metal or metalloid, a molar ratio of the at least one metal having the atomic number of 21, 39, or 57-71 to the at least one Group 4, 5, 6, 12, 13, 14, 15, or 16 metal or metalloid is at least 0.03:1, and a molar ratio of the at least one metal having the atomic number of 21, 39, or 57-71 to the Pt is at least 30:1, or (ii) wherein: the second catalyst comprises 0.025 wt % to 50 wt % of Cr based on a total weight of the second support, and the second support comprises SiO₂, ZrO₂, TiO₂, or a mixture thereof; and (III) feeding the effluent into a second conversion zone; and (IV) contacting the effluent with a solid oxygen carrier disposed within the second conversion zone to effect combustion of at least a portion of the molecular hydrogen to produce a conversion product comprising the one or more dehydrogenated hydrocarbons and water.

B2. The process of B1, wherein the first catalyst is present, and wherein the first catalyst further comprises an alkali metal element disposed on the first support.

B3. The process of B2, wherein the alkali metal element comprises Li, Na, K Rb, Cs, a combination thereof, or a mixture thereof.

B4. The process of B2 or B3, wherein the first catalyst comprises up to 5 wt % of the alkali metal based on the total weight of the first support.

B5. The process of any of B1 to B4, wherein the first catalyst is present, and wherein the molar ratio of the at least one metal having the atomic number of 21, 39, or 57-71 to the at least one Group 4, 5, 6, 12, 13, 14, 15, or 16 metal or metalloid is at least 0.03:1 to 2.7:1.

B6. The process of any of B1 to B5, wherein the first catalyst is present, and wherein the molar ratio of the at least one metal having the atomic number of 21, 39, or 57-71 to the Pt is at least 30 to 5000.

B7. The process of any of B1 to B6, wherein the first catalyst is present, and wherein the at least one compound comprising the at least one metal having the atomic number of 21, 39, or 57-71 or the at least one compound comprising the at least one metal having the atomic number of 21, 39, or 57-71 and the at least one Group 4, 5, 6, 12, 13, 14, 15, or 16 metal or metalloid is an oxide, a phosphate, a halide, a halate, a sulfate, a sulfide, a borate, a nitride, a carbide, an aluminate, an aluminosilicate, a silicate, a carbonate, metaphosphate, a selenide, a tungstate, a molybdate, a chromite, a chromate, a dichromate, or a silicide.

B8. The process of any of B1 to B7, wherein the first catalyst is present, and wherein the at least one metal having the atomic number of 21, 39, or 57-71 comprises at least one of Ce, Y, La, Sc, and Pr.

B9. The process of any of B1 to B8, wherein the first catalyst is present, and wherein the at least one Group 4, 5, 6, 12, 13, 14, 15, or 16 metal or metalloid comprises at least one of Zr, Al, Ti, and Si.

B10. The process of any of B1 to B9, wherein the first catalyst is present, and wherein the first support comprises a mixture of at least one compound comprising CeO₂, Y₂O₃, La₂O₃, Sc₂O₃, Pr₆O₁₁, and CePO₄, and at least one compound comprising Al₂O₃, SiO₂, ZrO₂, and TiO₂.

B11. The process of any of B1 to B10, wherein first catalyst is present, and wherein the first support comprises CeZrO₂, CeAlO₃, BaCeO₃, CePO₄, or a mixture thereof.

B12. The process of B1, wherein the second catalyst is present, and wherein the second catalyst further comprises an alkali metal element disposed on the second support.

B13. The process of B12, wherein the alkali metal element comprises Li, Na, K, Rb, Cs, a compound thereof, or a mixture thereof.

B14. The process of any of B1, B12, or B13, wherein the second catalyst is present, and wherein the second support further comprises at least one compound comprising at least one Group 5, 6, 12, 13, 15, or 16 metal or metalloid.

B15. The process of B14, wherein the at least one compound comprising the at least one Group 5, 6, 12, 13, 15, or 16 metal or metalloid is an oxide, a phosphate, a halide, a halate, a sulfate, a sulfide, a borate, a nitride, a carbide, an aluminate, an aluminosilicate, a silicate, a carbonate, metaphosphate, a selenide, a tungstate, a molybdate, a chromite, a chromate, a dichromate, or a silicide.

B16. The process of any of B1 to B15, wherein the solid oxygen carrier releases lattice oxygen during combustion of the molecular hydrogen.

B17. The process of any of B1 to B16, wherein the solid oxygen carrier is reduced from a first state SOₓC to a second state SO_{y}C during step (IV), wherein x is a positive number, y is a positive number, and y is <x, the process further comprising: (V) stopping feeding of the effluent into the second conversion zone; (VI) feeding an oxidant feed into the second conversion zone; (VII) reacting the solid oxygen carrier with a first portion of the oxidant to oxidize the solid oxygen carrier from the second state to a third state SO_{z}C, wherein z is a positive number, and wherein z is >y; (VIII) stopping feeding of the oxidant into the conversion zone; and (XI) repeating steps (III) to (V).

B18. The process of B17, wherein the process further comprises, after step (VIII) and before step (XI), the following steps: (VIIIb) feeding a reducing gas comprising molecular hydrogen, carbon monoxide, steam, or a mixture thereof into the second conversion zone; and (VIIIc) contacting the catalyst with the reducing gas to reduce at least a portion of the Pt from an oxidized state to a metallic state.

B19. The process of B17 or B18, wherein in step (II), coke is formed on the surface of the catalyst, and wherein in step (VII), a second portion of the oxidant combusts at least a portion of the coke on the surface of the catalyst.

B20. The process of any of B1 to B19, wherein the hydrocarbon-containing feed comprises a C₂ to C₁₈ alkane.

B21. The process of any of B1 to B20, wherein the hydrocarbon-containing feed comprises ethane, propane, butane, pentane, hexane, heptane, octane or a mixture thereof.

B22. The process of any of B1 to B21, wherein the hydrocarbon-containing feed comprises ethylbenzene, ethyl toluene, isopropyl benzene, diethylbenzenes, or a mixture thereof.

B23. The process of any of B1 to B22, wherein the hydrocarbon-containing feed contacts the first catalyst or the second catalyst in the first conversion zone at a weight hour space velocity of 0.01 hr⁻¹ to 300 hr⁻¹, at a temperature of 300° C. to 750° C., and under an absolute pressure of 10 kPa to 1,000 kPa.

B24. The process of any of B1 to B23, wherein the effluent contacts the solid oxygen carrier in the second conversion zone at a weight hour space velocity of 0.01 hr⁻¹ to 300 hr⁻¹, at a temperature of 300° C. to 750° C., and under an absolute pressure of 10 kPa to 1,000 kPa.

B25. The process of any of B1 to B24, wherein the first catalyst or the second catalyst and the solid oxygen carrier are each in the form of a plurality of particles.

B26. The process of any of B1 to B25, wherein the solid oxygen carrier comprises a metal in oxide form supported on a carrier, wherein the metal is selected from the group consisting of: an alkali metal, an alkaline earth metal, copper, chromium, molybdenum, vanadium, cerium, yttrium, scandium, tungsten, manganese, iron, cobalt, nickel, silver, bismuth, and a combination thereof.

B27. The process of B26, wherein the carrier is selected from the group consisting of: aluminum oxides, aluminum hydroxides, aluminum trihydroxide, boehmite, pseudo-boehmite, gibbsite, bayerite, transition aluminas, alpha-alumina, gamma-alumina, silica/alumina, silica, silicates, aluminates, calcium aluminate, barium hexaaluminate, calcined hydrotalcites, zeolites, zinc oxide, chromium oxides, magnesium oxides, zirconia oxides, and a combination thereof.

Various terms have been defined above. To the extent a term used in a claim is not defined above, it should be given the broadest definition persons in the pertinent art have given that term as reflected in at least one printed publication or issued patent. Furthermore, all patents, test procedures, and other documents cited in this application are fully incorporated by reference to the extent such disclosure is not inconsistent with this application and for all jurisdictions in which such incorporation is permitted.

While the foregoing is directed to embodiments of the present invention, other and further embodiments of the invention may be devised without departing from the basic scope thereof, and the scope thereof is determined by the claims that follow.

What is claimed is:

1. A process for dehydrogenating a hydrocarbon, comprising:
(I) feeding a hydrocarbon-containing feed comprising one or more of C₂-C₁₆ linear or branched alkanes, one or more of C₄-C₁₆ cyclic alkanes, one or more of C₈-C₁₆ alkyl aromatics, or a mixture thereof into a conversion zone;
(II) contacting the hydrocarbon-containing feed with a first catalyst comprising Pt disposed on a first support or a second catalyst comprising Cr disposed on a second support within the conversion zone to effect dehydrogenation of at least a portion of the hydrocarbon-containing feed to produce an effluent comprising one or more dehydrogenated hydrocarbons and molecular hydrogen,
(i) wherein:
the first catalyst comprises 0.025 wt % to 6 wt % of Pt based on a total weight of the first support,
the first support comprises at least one of: (i) at least one compound comprising at least one metal having an atomic number of 21, 39, or 57-71 and at least one compound comprising at least one Group 4, 5, 6, 12, 13, 14, 15, or 16 metal or metalloid, and (ii) at least one compound comprising at least one metal having an atomic number of 21, 39, or 57-71 and at least one Group 4, 5, 6, 12, 13, 14, 15, or 16 metal or metalloid,
a molar ratio of the at least one metal having the atomic number of 21, 39, or 57-71 to the at least one Group 4, 5, 6, 12, 13, 14, 15, or 16 metal or metalloid is at least 0.03:1, and
a molar ratio of the at least one metal having the atomic number of 21, 39, or 57-71 to the Pt is at least 30:1, or
(ii) wherein:
the second catalyst comprises 0.025 wt % to 50 wt % of Cr based on a total weight of the second support, and
the second support comprises SiO₂, ZrO₂, TiO₂, or a mixture thereof; and
(III) contacting the effluent with a solid oxygen carrier disposed within the conversion zone to effect combustion of at least a portion of the molecular hydrogen to produce a conversion product comprising the one or more dehydrogenated hydrocarbons and water.

2. The process of claim 1, wherein the first catalyst is present, and wherein the first catalyst further comprises an alkali metal element disposed on the first support, and wherein the alkali metal element comprises Li, Na, K Rb, Cs, a combination thereof, or a mixture thereof, and wherein the first catalyst comprises up to 5 wt % of the alkali metal based on the total weight of the first support.

3. The process of claim 1, wherein the first catalyst is present, and wherein the molar ratio of the at least one metal having the atomic number of 21, 39, or 57-71 to the at least one Group 4, 5, 6, 12, 13, 14, 15, or 16 metal or metalloid is at least 0.03:1 to 2.7:1.

4. The process of claim 1, wherein the first catalyst is present, and wherein the molar ratio of the at least one metal having the atomic number of 21, 39, or 57-71 to the Pt is at least 30 to 5000.

5. The process of claim 1, wherein the first catalyst is present, and wherein the at least one compound comprising the at least one metal having the atomic number of 21, 39, or 57-71 or the at least one compound comprising the at least one metal having the atomic number of 21, 39, or 57-71 and the at least one Group 4, 5, 6, 12, 13, 14, 15, or 16 metal or metalloid is an oxide, a phosphate, a halide, a halate, a sulfate, a sulfide, a borate, a nitride, a carbide, an aluminate, an aluminosilicate, a silicate, a carbonate, metaphosphate, a selenide, a tungstate, a molybdate, a chromite, a chromate, a dichromate, or a silicide.

6. The process of claim 1, wherein the first catalyst is present, and wherein the at least one metal having the atomic number of 21, 39, or 57-71 comprises at least one of Ce, Y, La, Sc, and Pr.

7. The process of claim 1, wherein the first catalyst is present, and wherein the at least one Group 4, 5, 6, 12, 13, 14, 15, or 16 metal or metalloid comprises at least one of Zr, Al, Ti, and Si.

8. The process of claim 1, wherein the first catalyst is present, and wherein the first support comprises a mixture of at least one compound comprising CeO₂, Y₂O₃, La₂O₃, Sc₂O₃, Pr₆O₁₁, and CePO₄, and at least one compound comprising Al₂O₃, SiO₂, ZrO₂, and TiO₂.

9. The process of claim 1, wherein first catalyst is present, and wherein the first support comprises CeZrO₂, CeAlO₃, BaCeO₃, CePO₄, or a mixture thereof.

10. The process of claim 1, wherein the second catalyst is present, and wherein the second catalyst further comprises an alkali metal element disposed on the second support, and wherein the alkali metal element comprises Li, Na, K, Rb, Cs, a compound thereof, or a mixture thereof.

11. The process of claim 1, wherein the second catalyst is present, and wherein the second support further comprises at least one compound comprising at least one Group 5, 6, 12, 13, 15, or 16 metal or metalloid.

12. The process of claim 11, wherein the at least one compound comprising the at least one Group 5, 6, 12, 13, 15, or 16 metal or metalloid is an oxide, a phosphate, a halide, a halate, a sulfate, a sulfide, a borate, a nitride, a carbide, an aluminate, an aluminosilicate, a silicate, a carbonate, metaphosphate, a selenide, a tungstate, a molybdate, a chromite, a chromate, a dichromate, or a silicide.

13. The process of claim 1, wherein the solid oxygen carrier is reduced from a first state SOₓC to a second state SO_{y}C during step (III), wherein x is a positive number, y is a positive number, and y is <x, the process further comprising:
(IV) stopping feeding of the hydrocarbon-containing feed into the conversion zone;
(V) feeding an oxidant feed into the conversion zone;
(VI) reacting the solid oxygen carrier with a first portion of the oxidant to oxidize the solid oxygen carrier from the second state to a third state SO_{z}C, wherein z is a positive number, and wherein z is >y;
(VII) stopping feeding of the oxidant into the conversion zone; and
(VIII) repeating steps (I) to (III).

14. The process of claim 13, wherein the process further comprises, after step (VII) and before step (VIII), the following steps:
(VIIb) feeding a reducing gas comprising molecular hydrogen, carbon monoxide, steam, or a mixture thereof into the conversion zone; and
(VIIc) contacting the catalyst with the reducing gas to reduce at least a portion of the Pt from an oxidized state to a metallic state.

15. The process of claim 12, wherein in step (II), coke is formed on the surface of the catalyst, and wherein in step (VI), a second portion of the oxidant combusts at least a portion of the coke on the surface of the catalyst.

16. The process of claim 1, wherein the hydrocarbon-containing feed contacts the first catalyst or the second catalyst in the conversion zone at a weight hour space velocity of 0.01 hr⁻¹ to 300 hr⁻¹, at a temperature of 300° C. to 750° C., and under an absolute pressure of 10 kPa to 1,000 kPa, and wherein the effluent contacts the solid oxygen carrier in the conversion zone at a weight hour space velocity of 0.01 hr⁻¹ to 300 hr⁻¹, at a temperature of 300° C. to 750° C., and under an absolute pressure of 10 kPa to 1,000 kPa.

17. The process of claim 1, wherein the first catalyst or the second catalyst and the solid oxygen carrier are each in the form of a plurality of particles, and wherein the first catalyst or the second catalyst and the solid oxygen carrier are mixed with one another within the conversion zone.

18. The process of claim 1, wherein the first catalyst or the second catalyst and the solid oxygen carrier are each in the form of a plurality of particles, and wherein the first catalyst or the second catalyst and the solid oxygen carrier are arranged in alternating layers within the conversion zone.

19. The process of claim 1, wherein the first catalyst or the second catalyst and the solid oxygen carrier are each in the form of a plurality of particles, and wherein the first catalyst or the second catalyst and the solid oxygen carrier are arranged in staged beds with respect to one another within the conversion zone.

20. A process for dehydrogenating a hydrocarbon, comprising:
(I) feeding a hydrocarbon-containing feed comprising one or more of C₂-C₁₆ linear or branched alkanes, one or more of C₄-C₁₆ cyclic alkanes, one or more of C₈-C₁₆ alkyl aromatics, or a mixture thereof into a first conversion zone;
(II) contacting the hydrocarbon-containing feed with a first catalyst comprising Pt disposed on a first support or a second catalyst comprising Cr disposed on a second support within the first conversion zone to effect dehydrogenation of at least a portion of the hydrocarbon-containing feed to produce an effluent comprising one or more dehydrogenated hydrocarbons and molecular hydrogen,
(i) wherein:
the first catalyst comprises 0.025 wt % to 6 wt % of Pt based on a total weight of the first support,
the first support comprises at least one of: (i) at least one compound comprising at least one metal having an atomic number of 21, 39, or 57-71 and at least one compound comprising at least one Group 4, 5, 6, 12, 13, 14, 15, or 16 metal or metalloid, and (ii) at least one compound comprising at least one metal having an atomic number of 21, 39, or 57-71 and at least one Group 4, 5, 6, 12, 13, 14, 15, or 16 metal or metalloid,
a molar ratio of the at least one metal having the atomic number of 21, 39, or 57-71 to the at least one Group 4, 5, 6, 12, 13, 14, 15, or 16 metal or metalloid is at least 0.03:1, and
a molar ratio of the at least one metal having the atomic number of 21, 39, or 57-71 to the Pt is at least 30:1, or
(ii) wherein:
the second catalyst comprises 0.025 wt % to 50 wt % of Cr based on a total weight of the second support, and
the second support comprises SiO₂, ZrO₂, TiO₂, or a mixture thereof; and
(III) feeding the effluent into a second conversion zone; and
(IV) contacting the effluent with a solid oxygen carrier disposed within the second conversion zone to effect combustion of at least a portion of the molecular hydrogen to produce a conversion product comprising the one or more dehydrogenated hydrocarbons and water.
